(19) United States
(12) Patent Application Publication
(10) Pub. No.: US 2006/0036300 A1
(43) Pub. Date: Feb. 16, 2006

(54) Method for lypolisis

(75) Inventor: Michael Kreindel, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Syneron Medical Ltd., Yokneam Illit (IL)

(21) Appl. No.: 10/918,735

(22) Filed: Aug. 16, 2004

(51) Int. Cl. A61F 7/00; A61F 2/00; A61F 7/12

(52) U.S. Cl. 607/99; 607/101; 607/113

(57) ABSTRACT

A method of lipolysis. The method comprises deforming a region of skin so that the region of skin protrudes from surrounding skin. One or more radio frequency (RF) electrodes are positioned on the protruding region of skin so as to generate an electrical current through adipose tissue in the protruding region of skin when a voltage is applied to the electrode or electrodes. A voltage is then applied to the electrode or electrodes so as to deliver sufficient RF energy to the protruding region of skin to damage subcutaneous adipose tissue. The method of the invention may be used, for example, to achieve a reduction in body weight, cellulite reduction, loose skin reduction, wrinkle treatment, body surface tightening, skin tightening, and collagen remodeling.

### FIELD OF THE INVENTION

The invention relates to methods for treating adipose tissue.

### BACKGROUND OF THE INVENTION

Skin tissue consists of an outer epidermal layer overlying a dermal layer that is in contact with a layer of subcutaneous adipose tissue. Excess adipose tissue is responsible for such medical problems as obesity, cellulites, loose skin, and wrinkles. By destroying the adipose cells, the appearance of the outer layer of the skin can be improved. Damaged adipose tissue is evacuated from the body by the lymphatic system. The destruction of adipose tissue in the sub-dermal layer often provides the following medical and cosmetic solutions: weight reduction, cellulite reduction, loose skin reduction, deep wrinkle reduction and body re-contouring. Reduction of the fat content may also cause skin tightening. Wrinkles are created in skin due to the breakage of collagen fibers and to the penetration of fat into the dermal layer of the skin.

Most existing wrinkle treatment methods target the collagen but do not have a significant effect on deep wrinkles. Radio frequency (RF) energy has been actively used for the treatment of epidermal and dermal layers of the skin. For example U.S. Pat. No. 6,749,626 describes the use of RF energy for collagen formation in the dermis. U.S. Pat. No. 6,241,753 describes a method for collagen scar formation. U.S. Pat. Nos. 6,470,216, 6,438,424, 6,430,446 and 6,461,378 disclose methods and apparatuses for destroying the collagen matrix using RF, cooling and a special electrode structure that smoothes the skin surface. U.S. Pat. Nos. 6,453,202, 6,405,090, 6,381,497, 6,311,090, 5,871,524 and 6,425,912 describe methods and apparatuses for delivering RF energy to the skin using membrane structure. U.S. Pat. Nos. 6,453,202 and 6,425,912 describe method and apparatus for delivering RF energy to the skin using dielectric electrodes. U.S. Pat. Nos. 6,381,498, 6,377,855, 5,919,219, 5,948,011, 5,755,753 describe methods of collagen contraction using RF energy, and a reverse temperature gradient on the skin surface.

U.S. Pat. Nos. 6,378,380, 6,377,854 and 5,660,836 describe methods of lyposculptering using RF energy and external cooling to affect the collagen inside the adipose tissue.

Another method to reduce and redistribute adipose issue is skin massaging. This method is based on improving of blood circulation and increasing fat metabolism. U.S. Pat. No. 6,662,054 describes a method for skin massaging in combination with non-aggressive RF heating for increasing skin and fat metabolism.

U.S. Pat. No. 6,273,884 to Altshuler et al. discloses simultaneous application of optical energy and negative pressure to the skin in order to treat a skin defect. This method is limited by the light penetration depth, which does not exceed a 1-2 millimeters.

U.S. Pat. No. 5,143,063 describes a method based on thermal destruction of fat using the focusing of microwave or ultrasound energy in adipose tissue. But both types of energy are very expensive and its safety limitations are not clear.

The above mentioned methods attempt to solve the problems created by excess adipose tissue such as body contouring, loose skin, and deep wrinkles, by contracting the superficial collagen tissue. These methods are limited in their penetration depth. A more effective and longer lasting result would be achieved by directly affecting the adipose tissue. However, in order to reach the sub-dermal layer adipose tissue, it is necessary to deliver RF current into the fat tissue to a depth of over 2 mm without damaging the skin. Furthermore, the amount of energy and duration of the energy application should be high enough to create adipose tissue necrosis.

### SUMMARY OF THE INVENTION

1. The present invention provides a method for delivering RF energy sufficiently deep below the skin surface so as to generate a heating of the deep skin layer that is strong enough to destroy fat cells. In accordance with the invention, a region of skin is deformed so that the region protrudes out from surrounding skin. The deformed skin preferably protrudes above the periphery of the region to a height of 1 to 30 mm.

One or more RF electrodes are then applied to the skin protrusion in order to direct the RF current through the skin protrusion. Deforming of the skin can be done by applying vacuum suction to the skin surface or by pinching the skin surface. Alternatively, deforming the skin can be done by applying a pressure to the periphery of the treated skin region that is higher than a pressure applied at the interior of the region.

In one preferred embodiment of the invention, a bipolar RF system is used. In this embodiment, the distance between the two electrodes preferably exceeds 4 mm. As the distance between the electrodes increases, the electrical current divergence is stronger so that deeper layers of tissue can be heated.

In another preferred embodiment, a uni-polar RF system is used. In this embodiment, the area of the RF electrode preferably has an area exceeding 5 mm². With a large electrode size the divergence of the electrical current is low at depths up to the electrode size.

Necrosis of tissue is a function of temperature of the tissue and the time duration during which the temperature is maintained. The range of temperatures in which adipose tissue necrosis can be achieved varies from 45° C. and up to 100° C., when boiling of water occurs. The practical duration of RF energy application can vary from 0.01 up to 10 sec. During this time, RF energy can be delivered continuously or in a pulsed manner. Longer pulses of treatment may limit the total treatment time.

The density of an RF current is always higher around the surface of the RF electrode applied to the skin surface. In order to avoid overheating the skin, the skin may be cooled. Cooling may be applied prior the RF energy application or/and simultaneously. The surface of the skin can be cooled using a cooled liquid or by cooling of the electrode surface. The cooling depth (d) depends on the cooling application time (t), and can be estimated from the following equation:
d=\sqrt{4αt},
where α is the diffusivity of the tissue, which is similar to liquid and is about 1.4×10⁻⁷ m²/s. Thus, with a cooling duration longer then 2.5 sec, all layers of the skin up to about 2 mm below the surface will be cooled, so that the temperature of the adipose layer will be higher than the temperature of the dermis and epidermis. In most cases, the thickness of the dermis over the adipose layer does not exceed 1 mm. In this case, a cooling time of 1.5 sec is enough to cool the skin.

The RF electrodes may be made from metal or a semi-conductive material.

In one embodiment the electrodes are covered with a dielectric material. A liquid or gel medium can be used for electrical and thermal coupling between the applicator and the body surface.

The RF current may be combined with optical energy where the RF energy is used for heating a deeper layer while the light is used for subcutaneous fat distruction. Infrared light in the range of 700 nm to 1500 nm is preferably used in order to penetrate inside the tissue to a depth of about two millimeters. A diode laser produces optical radiation in this range and can be used in combination with RF energy. In another embodiment, filtered broad-spectrum light produced by a flash lamp can be used.

The parameters of the RF may be adjusted for selective destruction of adipose tissue, which is less effected by cooling by blood perfusion due to the lower blood vessel contents of adipose tissue. The selective destruction of adipose tissue may be combined with collagen restructuring inside the skin.

The method of the invention may be used, for example, to achieve a reduction in body weight, cellulite reduction, loose skin reduction, wrinkle treatment, body surface tightening, skin tightening, and collagen remodeling. Thus, in its first aspect, the invention provides a method of lipolysis comprising, for each of one or more regions of skin:
deforming the skin so that the region of skin protrudes from surrounding skin;
positioning one or more radio frequency (RF) electrodes on the protruding region of skin, the electrodes being positioned on the protruding region of skin so as to generate an electrical current through adipose tissue in the protruding region of skin when a voltage is applied to the electrode or electrodes; and
applying a voltage to the electrode or electrodes so as to deliver sufficient RF energy to the protruding region of skin to damage subcutaneous adipose tissue.

In its second aspect, the invention provides a method of lipolysis comprising, for each of one or more regions of the skin:
applying two or more RF electrodes to the region of skin, the electrodes having a distance between electrodes sufficient to deliver a quantity of RF energy to the adipose tissue damaging to the adipose tissue; and
applying a quantity of RF energy sufficient to damage the adipose tissue.

In its third aspect, the invention provides a method of lipolysis comprising, for each of one or more regions of the skin:
applying an RF electrode having a surface area sufficient to deliver a damaging amount of energy to the sub-dermal layer; and
applying sufficient quantity of RF energy to the region of skin to damage adipose tissue.

In its fourth aspect, the invention provides a method of lipolysis comprising, for each of one or more regions of skin:
applying two or more RF electrodes to the region with a distance between two electrodes sufficient to deliver an amount of energy damaging to adipose disuse;
applying light energy having a spectral range such that at least part of the radiation penetrates beneath the dermal layer; and
applying sufficient quantity of RF and light energy energy to damage the adipose tissue.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows an applicator for application of Rf energy to a protruding region of skin in accordance with one embodiment of the invention;

FIG. 2 shows an applicator for applying RF energy to a protruding region of skin in accordance with another embodiment of the invention;

FIG. 3 shows a unipolar RF applicator for applying RF energy to a protruding region of skin in accordance with a third embodiment of the invention; and

FIG. 4 shows an applicator for applying RF energy to a protruding region of skin in accordance with a fourth embodiment of the invention.

### DETAILED DESCRIPTION OF PREFERRED EMBODIMENTS

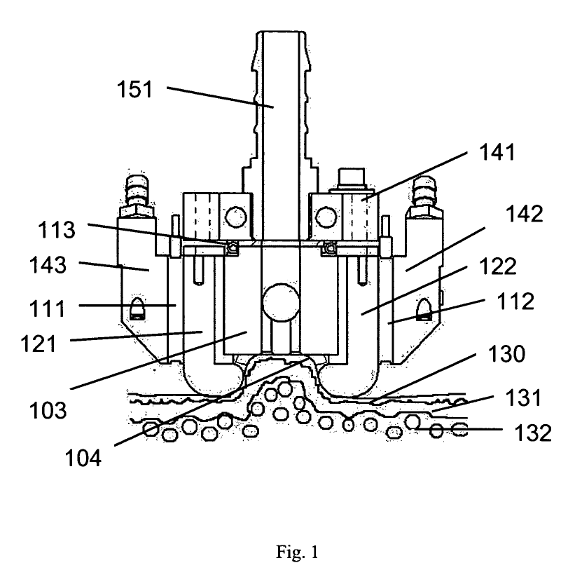

Referring first to FIG. 1, an applicator 100 is shown for applying negative pressure and RF energy to skin in accordance with one embodiment of the invention. The applicator 100 is configured to be connected to an RF generator (not shown), such as the Rf generator disclosed in Applicant's copending U.S. patent application Ser. No. 10/105,885 filed on Mar. 3, 2002, which is incorporated herein in its entirety by reference. The applicator 100 is configured to be applied to a region of the skin of an individual to be treated. The applicator includes an applicator body 103 formed from a material having a high thermal conductivity and enclosing the bell-shaped chamber 104. The bell-shaped chamber 104 is open on the bottom so that when applied to a region of skin, the skin is in contact with the bell-shaped chamber 104. The area of skin in contact with the interior chamber 104 is preferably between 0.5 to 20 cm². The skin tissue includes an epidermal layer 131 and a dermal layer 132 overlaying a layer of subcutaneous adipose tissue 132. The depth of the chamber 104 defines the height of RF energy delivery. The height of the chamber 104 is preferably from 2 mm to 20 mm. Deeper heating may be used for the treatment of such areas as the buttocks, abdomen and thighs requiring a large treatment area. In the embodiment of FIG. 1, the depth of the chamber 104 is 12 mm for heating a thick layer of fat at a depth from 2 mm to 15 mm.

The applicator 100 further includes RF electrodes 121 and 122 that are embedded in the body 103 and are configured to apply RF energy to the region of skin to be treated. Cooling elements 111, 112 are attached to the electrodes 121, 122 and the body 103 to provide cooling of the skin surface. In the embodiment of FIG. 1, the cooling elements 111, 112 and 113 are thermo-electric coolers. In this case, the hot sides of the thermo-electric coolers are cooled by circulating liquid flowing through heat exchangers 141, 142 and 143. Alternatively, a cooling effect may be attained by circulation of a pre-cooled liquid or by use of a cryogen spray.

The skin is made to protrude into the chamber 104 using vacuum suction applied through the inlet 151. The skin protrusion is thus between the two electrodes 121 and 122.

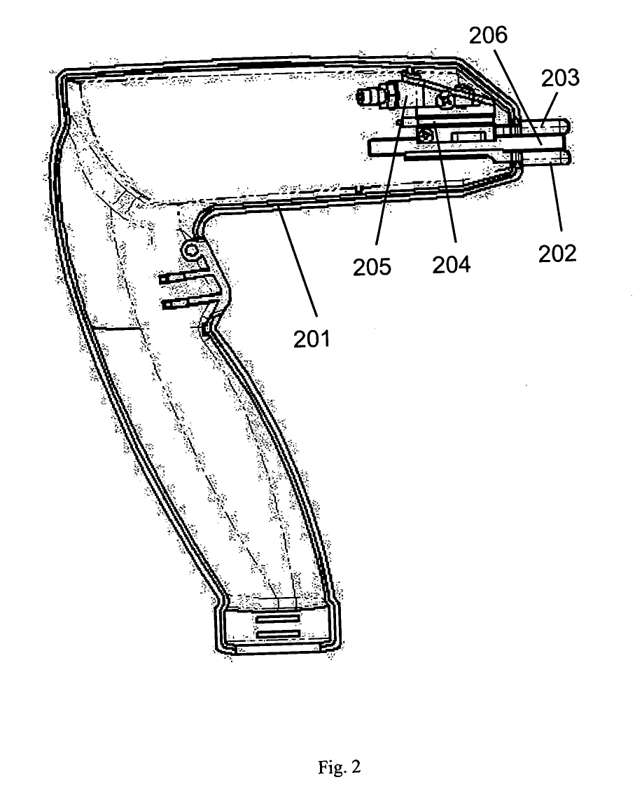

FIG. 2 shows an applicator 201 in accordance with another embodiment of the invention having two electrodes 202 and 203. The surface of the electrodes 202 and 203 are rounded to provide a uniform distribution of electrical current over the area of contact of the electrodes with the skin. In this embodiment, the distance between the electrodes 202 and 203 is chosen according to the desired heating depth and is in the range of 4 mm to 20 mm. In order to avoid overheating of the skin surface in the area of contact with the electrodes 202, 203, the electrode surface is cooled by a thermoelectric cooler 204. The hot side of the thermoelectric cooler is cooled by a circulating liquid flowing through the heat exchanger 205. A spacer 206 between electrodes 202 and 203 is made from a heat conductive and electrical isolating material. For example, a ceramic material or sapphire may be used.

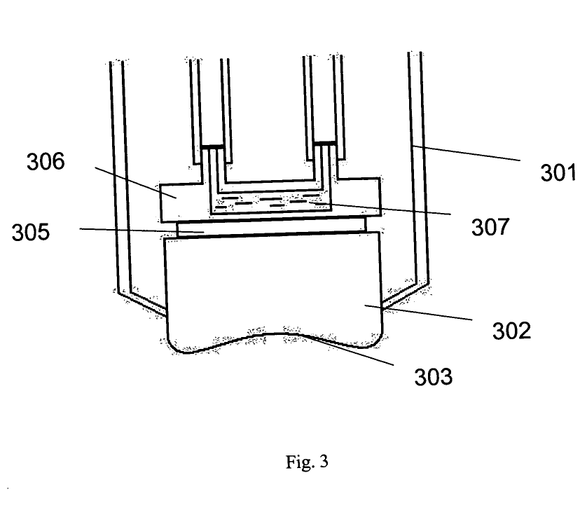

FIG. 3 shows an applicator 301 in accordance with another embodiment of the invention. The applicator 301 has a single electrode 302. The electrode 302 has a surface 303, which is coupled to the skin surface. In the current embodiment of FIG. 3 the size of the electrode is preferably about one square centimeter but it can be smaller but preferably not less than 3 mm². The surface of the electrode 301 may be covered by a thin layer of dielectric material for capacitive coupling of RF energy to the treated tissue. The surface of the electrode may be curved for better coupling to the skin surface and for optimal energy delivery to the sub-dermal layer of adipose tissue. In order to avoid overheating of the skin surface in the area of contact with electrodes 302, the electrode surface is cooled by thermoelectric coolers 305. The hot side of thermoelectric cooler is cooled by a cooled liquid such as water 307 circulating through a heat exchanger 306. Alternatively, the opposite side of the electrode may be cooled by cooling media such as a cooled liquid or cryogen spray. In this case, the electrode is preferably designed from a metal foil in order to enhance heat transfer from the skin surface to the cooling media. The electrode material is preferably a metal having high thermal conductivity such as copper or copper alloy, aluminum, silver or gold.

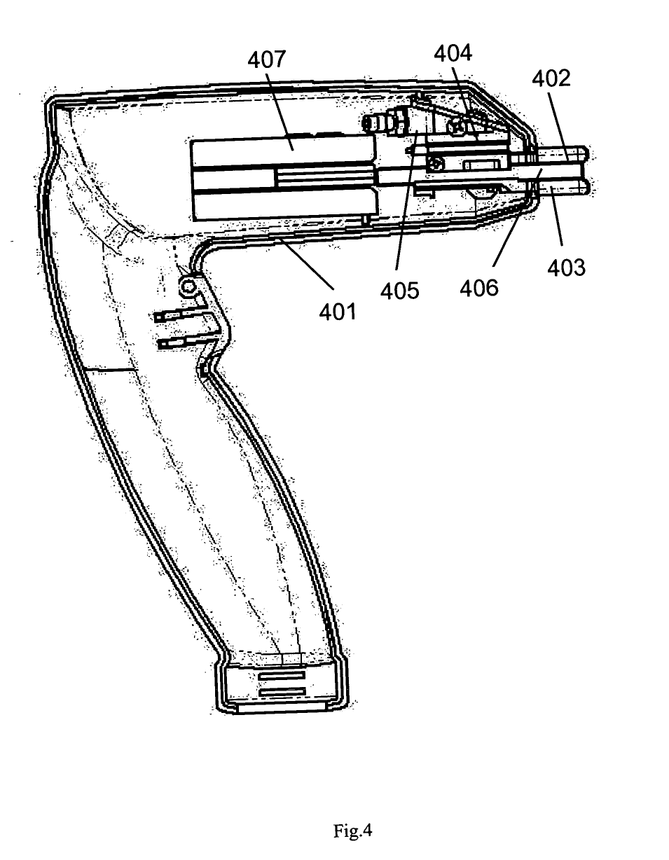

FIG. 4 shows an applicator 401 in accordance with another embodiment of the invention. The applicator 401 has two electrodes 402 and 403. The electrode surface is rounded to provide a uniform distribution of electrical current over the area of contact with the skin. In this embodiment, the distance between the electrodes 402 and 403 is chosen according to desired heating depth and is in the range of 4 mm to 30 mm. Light guide 406 between electrodes 402, 403 is made from a heat conductive and transparent material, such as quartz or sapphire. The light guide 406 delivers optical energy produced by light source 407. The light source 407 may be, for example, a diode laser, flash lamp, alexandrite laser, Nd:Yag laser or other light source producing radiation in the range of 600 nm to 2000 nm. The light should have an intensity so as to penetrate into the skin deep enough to reach the adipose tissue. In order to avoid overheating of the skin surface in the area of contact with electrodes 402, 403 and light guide 406, the electrodes and light guide surface are cooled by a thermoelectric cooler 404. The hot side of thermoelectric cooler is cooled by a circulating liquid flowing through the heat exchanger 405. Light energy produced by the light source 407 is delivered through the light guide 406 to the same region of skin that is treated with RF current.

Using the system of the invention to treat subcutaneous adipose tissue, the following exemplary parameter values of RF energy may be used:
RF frequency: 0.1-30 MHz.
Average output power: from about 1 to about 3000 W.
Delivered energy should exceed 20 J/cm³.
Energy delivery time should be longer than one millisecond to avoid skin overheating at the electrode contact but the optimal energy delivery time is longer than 200 ms. The energy may be delivered during the energy delivery time continuously or with sequence of pulses.
The optimal cooling temperature of the electrode should be in the range of ° C. to 15° C.

The parameters of optical source 407 shown in the FIG. 4 may be as follows:
Light fluence 2 J/cm² to 200 J/cm².
Light spectrum is in the range of 600 nm to 1500 nm.
Optical energy delivery time can be varied from 1 ms to 5 sec, but the optimal range is from 100 msec to 2 sec. The optical energy may be delivered continuously or by train of the pulses during the energy delivery time.

1. A method of lipolysis comprising, for each of one or more regions of skin:
(a) deforming the skin so that the region of skin protrudes from surrounding skin;
(b) positioning one or more radio frequency (RF) electrodes on the protruding region of skin, the electrodes being positioned on the protruding region of skin so as to generate an electrical current through adipose tissue in the protruding region of skin when a voltage is applied to the electrode or electrodes; and
(c) applying a voltage to the electrode or electrodes so as to deliver sufficient RF energy to the protruding region of skin to damage subcutaneous adipose tissue.

2. The method according to claim 1 further comprising cooling the skin surface.

3. The method according to claim 1 wherein the step of deforming the skin involves applying a negative pressure to the skin.

4. The method according to claim wherein one RF electrode is applied to the protruding region of skin.

5. The method according to claim 4 wherein the RF electrode has a surface area of at least 5 mm².

6. The method according to claim 1 wherein two RF electrodes are applied to the protruding region of skin.

7. The method according to claim 6 wherein the protruding region of skin is positioned between the two electrodes applied to the protruding region of skin.

8. The method according to claim 6 wherein the two electrodes are separated by a distance of between 4 and 40 mm.

9. The method according to claim 1 wherein the frequency of the RF energy is from 0.1 to 30 MHz.

10. The method according to claim 1 wherein the power of the RF energy is from 5 to 2000 W.

11. The method according to claim 1 wherein a conductive liquid media is applied to the skin surface for coupling between an electrode and the skin surface.

12. The method according to claim 1 wherein one or more of the electrodes has a curved surface.

13. The method according to claim 1 wherein a surface the electrode contacting the skin is covered by a dielectric material.

14. The method according to claim 1 wherein an electrode surface is made from a metal foil.

15. The method according to claim 1 further comprising applying light energy to the skin having wherein at least a portion of a spectral range of the light penetrates beneath the dermal layer

16. The method according to claim 15 wherein the light has a spectrum, at least a portion of the spectrum being in the range of 600 nm to 2000 nm.

17. The method according to claim 15 wherein the light is produced by a light source selected from the group comprising a flash lamp, bulb lamp, diode laser, alexandrite laser, or Nd:YAG laser.

18. The method according to claim 1 wherein the step of deforming a skin region involves applying a pressure on the periphery of the skin region that is higher that a pressure applied to the interior of the region.

19. The method according to claim 1 wherein a portion of a deformed skin region is protrudes above the periphery of the region to a height of 1 to 30 mm.

20. The method according to claim 1 further comprising collagen remodeling.

21. Use of the method according to claim 1 in a process selected from the group comprising:
(a) reducing body weight;
(b) cellulite reduction;
(c) loose skin reduction;
(d) wrinkle treatment;
(e) body surface tightening;
(f) skin tightening; and
(g) collagen remodeling.

22. A method of lipolysis comprising, for each of one or more regions of the skin:
(a) applying two or more RF electrodes to the region of skin, the electrodes having a distance between electrodes sufficient to deliver a quantity of RF energy to the adipose tissue damaging to the adipose tissue; and
(b) applying a quantity of RF energy sufficient to damage the adipose tissue.

23. The method according to claim 22 further comprising cooling the skin surface.

24. The method according to claim 22 wherein two RF electrodes are applied to the protruding region of skin.

25. The method according to claim 25 wherein the two electrodes are separated by a distance of between 4 and 40 mm.

26. The method according to claim 22 wherein the frequency of the RF energy is from 0.1 to 30 MHz.

27. The method according to claim 22 wherein the power of the RF energy is from 5 to 2000 W.

28. The method according to claim 22 wherein a conductive liquid media is applied to the skin surface for coupling between an electrode and the skin surface.

29. The method according to claim 22 wherein one or more of the electrodes has a curved surface.

30. The method according to claim 22 wherein a surface the electrode contacting the skin is covered by a dielectric material.

31. The method according to claim 22 wherein an electrode surface is made from a metal foil.

32. The method according to claim 22 further comprising applying light energy to the skin having wherein at least a portion of a spectral range of the light penetrates beneath the dermal layer

33. The method according to claim 32 wherein the light has a spectrum, at least a portion of the spectrum being in the range of 600 nm to 2000 nm.

34. The method according to claim 32 wherein the light is produced by a light source selected from the group comprising a flash lamp, bulb lamp, diode laser, alexandrite laser, or Nd:YAG laser.

35. The method according to claim 22 further comprising collagen remodeling.

36. Use of the method according to claim 22 in a process selected from the group comprising:
(a) reducing body weight;
(b) cellulite reduction;
(c) loose skin reduction;
(d) wrinkle treatment;
(e) body surface tightening;
(f) skin tightening; and
(g) collagen remodeling.

37. A method of lipolysis comprising, for each of one or more regions of the skin:
(a) applying an RF electrode having a surface area sufficient to deliver a damaging amount of energy to the sub-dermal layer; and
(b) applying sufficient quantity of RF energy to the region of skin to damage adipose tissue.

38. The method according to claim 37 further comprising cooling the skin surface.

39. The method according to claim 37 wherein the RF electrode has a surface area of at least 5 mm².

40. The method according to claim 37 wherein the frequency of the RF energy is from 0.1 to 30 MHz.

41. The method according to claim 37 wherein the power of the RF energy is from 5 to 2000 W.

42. The method according to claim 37 wherein a conductive liquid media is applied to the skin surface for coupling between an electrode and the skin surface.

43. The method according to claim 37 wherein the electrode has a curved surface.

44. The method according to claim 37 wherein a surface the electrode contacting the skin is covered by a dielectric material.

45. The method according to claim 37 wherein an electrode surface is made from a metal foil.

46. The method according to claim 37 further comprising applying light energy to the skin having wherein at least a portion of a spectral range of the light penetrates beneath the dermal layer

47. The method according to claim 46 wherein the light has a spectrum, at least a portion of the spectrum being in the range of 600 nm to 2000 nm.

48. The method according to claim 46 wherein the light is produced by a light source selected from the group comprising a flash lamp, bulb lamp, diode laser, alexandrite laser, or Nd:YAG laser.

49. The method according to claim 37 further comprising collagen remodeling.

50. Use of the method according to claim 1 in a process selected from the group comprising:
(a) reducing body weight;
(b) cellulite reduction;
(c) loose skin reduction;
(d) wrinkle treatment;
(e) body surface tightening;
(f) skin tightening; and
(g) collagen remodeling.

51. A method of lipolysis comprising, for each of one or more regions of skin:
(a) applying two or more RF electrodes to the region with a distance between two electrodes sufficient to deliver an amount of energy damaging to adipose disuse;
(b) applying light energy having a spectral range such that at least part of the radiation penetrates beneath the dermal layer; and
(c) applying sufficient quantity of RF and light energy energy to damage the adipose tissue.

52. The method according to claim 51 further comprising cooling the skin surface.

53. The method according to claim 51 wherein an RF electrode has a surface area of at least 5 mm².

54. The method according to claim 51 wherein two RF electrodes are applied to the protruding region of skin.

55. The method according to claim 51 wherein two electrodes are separated by a distance of between 4 and 40 mm.

56. The method according to claim 51 wherein the frequency of the RF energy is from 0.1 to 30 MHz.

57. The method according to claim 51 wherein the power of the RF energy is from 5 to 2000 W.

58. The method according to claim 51 wherein a conductive liquid media is applied to the skin surface for coupling between an electrode and the skin surface.

59. The method according to claim 51 wherein one or more of the electrodes has a curved surface.

60. The method according to claim 51 wherein a surface the electrode contacting the skin is covered by a dielectric material.

61. The method according to claim 51 wherein an electrode surface is made from a metal foil.

62. The method according to claim 51 wherein the light has a spectrum, at least a portion of the spectrum being in the range of 600 nm to 2000 nm.

63. The method according to claim 51 wherein the light is produced by a light source selected from the group comprising a flash lamp, bulb lamp, diode laser, alexandrite laser, or Nd:YAG laser.

64. The method according to claim 51 further comprising collagen remodeling.

65. Use of the method according to claim 51 in a process selected from the group comprising:
(a) reducing body weight;
(b) cellulite reduction;
(c) loose skin reduction;
(d) wrinkle treatment;
(e) body surface tightening;
(f) skin tightening; and
(g) collagen remodeling.
